(19) United States
(12) Patent Application Publication
(10) Pub. No.: US 2008/0058583 A1
(43) Pub. Date: Mar. 6, 2008

(54) ARTICLES EFFECTING WELLNESS

(76) Inventor: Patricia Mohan, Toronto (CA)

Correspondence Address: BERESKIN AND PARR, 40 KING STREET WEST, BOX 401, TORONTO, ON, M5H 3Y2, US

(21) Appl. No.: 11/468,800

(22) Filed: Aug. 31, 2006

(51) Int. Cl. A61M 21/02

(52) U.S. Cl. 600/26

(57) ABSTRACT

Articles displaying a wellness indicia. The wellness indicia may include a Reiki symbol or combination of Reiki symbols. Different articles may display the wellness indicia, such as socks, gloves, hats, touques, scarves, neck rolls, lingerie, support wraps, blankets, collars, animal clothing, bedding, wallpaper, lampshades, sheets, pillow cases.

### FIELD OF THE INVENTION

The present invention relates generally to the field alternative medical treatments.

### BACKGROUND OF THE INVENTION

Reiki is a vibrational, or subtle energy therapy most commonly used either with or without touch. It is a healing modality that is included in the biofield therapies which are generally considered to be low-risk intervention. The National Institutes of Health Center for Complimentary and Alternative Medicine (NCCAM) has classified energy medicine therapies into 2 basic categories: biofield therapies and bioelectromagnetic-based therapies.

Biofield modalities are defined as those therapies intended to affect the energy fields that surround the human body. These are Reiki, Qigong, and Therapeutic touch. A growing number of medical and health care workers are adding Reiki to their repertoire for dealing with their clients. Biofield therapy is a healing modality in which the energy targets the receptor sites in the body corresponding to the endocrine glands and organs of the body (see meridian charts) releasing the energy blockages and resulting in improved health.

We have become aware of “life force energy” as the cause of our health and the lack as the cause of illness. Science is studying the “life force” flow and has become aware of energy blockages becoming illness, and when we alter our thought patterns we can often affect these blockages and then return to health. Reiki helps to transform these energy blocks, some of them have been with us for our whole life due to a shock when young or a fear that may seem irrational to the adult mind.

Our bodies are composed of physical elements such as muscles, bones, nerves, arteries, organs, glands, etc.; each has subtle energy bodies which surround our physical body and assist us in processing our thoughts and emotions. Our energy bodies have energy centers called charkas, which work somewhat like valves that allow life force to circulate through the physical, mental, and emotional bodies. We also have that energy flowing through our meridians and or nadis all of which become energy pathways to organs and glands. The American Institute of Stress estimates that 75%-95% of all visits to doctors are the results of reactions to stress.

It stands to reason that to reduce stress alone within the body it would increase health and longevity. The chakras when balanced become a source of useable energy for the nerve, cellular and hormonal systems within the physical body. The conduits or subtle “pathways” which facilitate this interchange between psychological and physical energy are called nadis (meridians). Various scientific studies have finally given credibility to the existence of the chakras. Dysfunction at the level of the chakras and nadis is directly reflected in pathological disorders within the nervous system/endocrine system which, in turn, will affect the whole body/mind. The physical, mental and spiritual aspects of the body work together and are interconnected. It is, therefore, important to remember that any disorder at the physiological level will always be, to some extent, reflected at the subtler levels.

Reiki has several effects: it increases the vibrational frequency of the body, releases blocks, sensations of deep relaxation and restored vitality. Reiki typically involves the tracing of symbols in the air through hand gestures. The symbols are like switches in Reiki treatments and their power is increased with intent but not necessary. In Reiki the symbols are the tools and function as the conductors or the vibrational energies running from them into the organism using them.

Reiki can be performed in person or in distance and either way the treatment has results.

The Reiki symbols are Sanskrit-derived Japanese forms. Sanskrit is the mother-tongue of all other languages. It is the language of the Vedas; the oldest writings known to man. The Vedas stated that Sanskrit is the language of the spirit world.

Reiki is typically performed by accredited practitioners.

Accordingly, the inventor has recognized a need for apparatus for providing the healing benefits of Reiki to individuals, without the need for accredited practitioners.

### SUMMARY OF THE INVENTION

This invention is directed toward an article intended to be worn on a body. The article includes a wellness indicia displayed thereon.

The wellness indicia typically comprises at least one Reiki symbol selected from the following set of Reiki symbols: Cho Ku Rei, Cho Ku Rei backwards, Sei He Ki, Sei He Ki, Sei He Ki backwards, Sei He Ki upside down, Sei He Ki upside down and backwards, Hon Sha Ze Sho Nen, Dai Ko Myo, Raku, Halu, Zonar, Harth, Mara, Johre, Len So My, Lon Say, Yod, Shanti, Sati, Om, Hung, palm Di Ko Mio, Sa, To, Ga, Ozza.

The article may be one or more articles of clothing such as socks, shirts, pants, gloves, hats, touques, scarves, neck rolls and lingerie, and may also be support wraps, blankets, collars, animal clothing, bedding, wallpaper, lampshades, sheets, pillow cases.

### BRIEF DESCRIPTION OF THE DRAWINGS

The present invention will now be described, by way of example only, with reference to the following drawings, in which like reference numerals refer to like parts and in which:

FIGS. 1A-1V illustrate various Reiki symbols which may be displayed as wellness indicia in accordance with the present invention.

FIGS. 2A-2M illustrate various configurations or combinations of Reiki symbols which may be displayed as wellness indicia in accordance with the present invention.

FIG. 3 is a reflexology chart of a human foot.

FIG. 4 is a meridian chart of a human body.

FIG. 5 illustrates a plurality of example articles, socks, made in accordance with the subject invention.

FIG. 6 illustrates a plurality of example articles, insoles, made in accordance with the subject invention.

FIG. 7 illustrates a plurality of example articles, including gloves, mitts, a hat, a toque, scarves, and a neck roll, all made in accordance with the subject invention.

FIGS. 8-11 illustrate several articles in the form of outfits, having matching shirts and pants made in accordance with the present invention.

FIG. 12 illustrates various other examples of articles made in accordance with the present invention and typically intended for use with animals.

FIG. 13 illustrates yet other examples of articles made in accordance with the present invention, including a sheet or bedspread, a lamp shade, a pillowcase, and a blanket

### DESCRIPTION OF THE PREFERRED EMBODIMENTS

Referring to FIGS. 5, illustrated therein is an example of a plurality of articles made in accordance with the subject invention. The figure illustrates a plurality of articles 10 (in this instance, socks 11) from both a side perspective view 11^{A} together with a bottom view 11^{B}, made in accordance with the subject invention. Each sock 11 displays a wellness indicia 12. The socks 11 are intended to be worn and used by an individual. The wellness indicia 12 may comprise a single symbol 14 or a combination 16 of a plurality of symbols as will be discussed in greater detail below.

### The Reiki Symbols

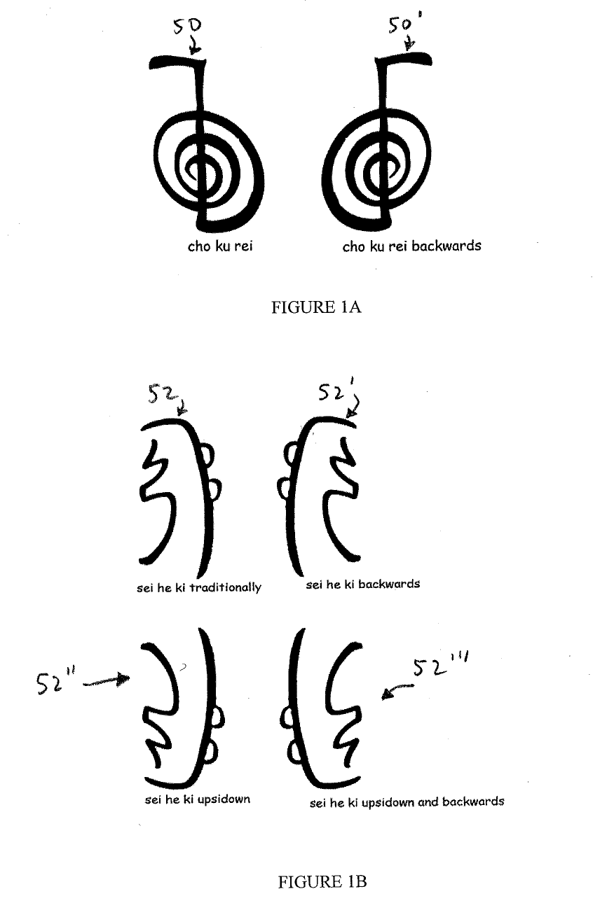

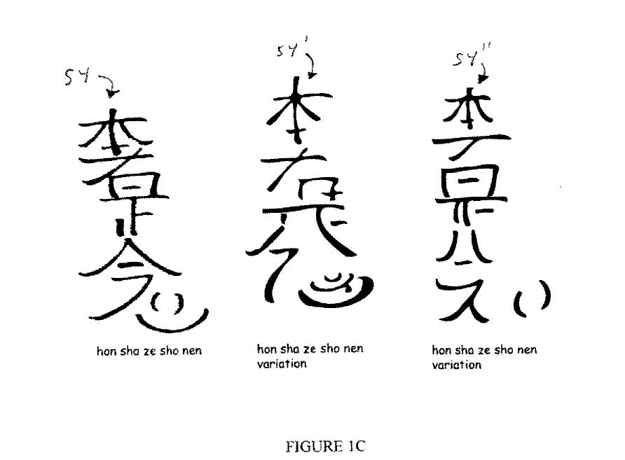

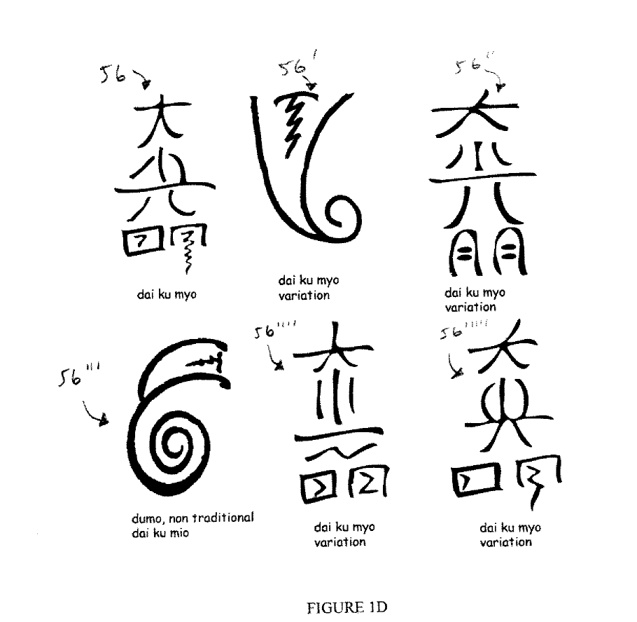

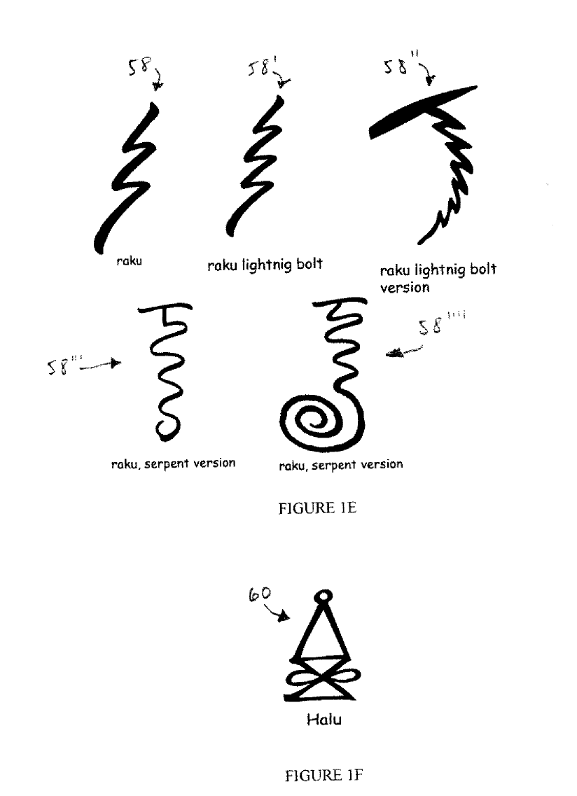

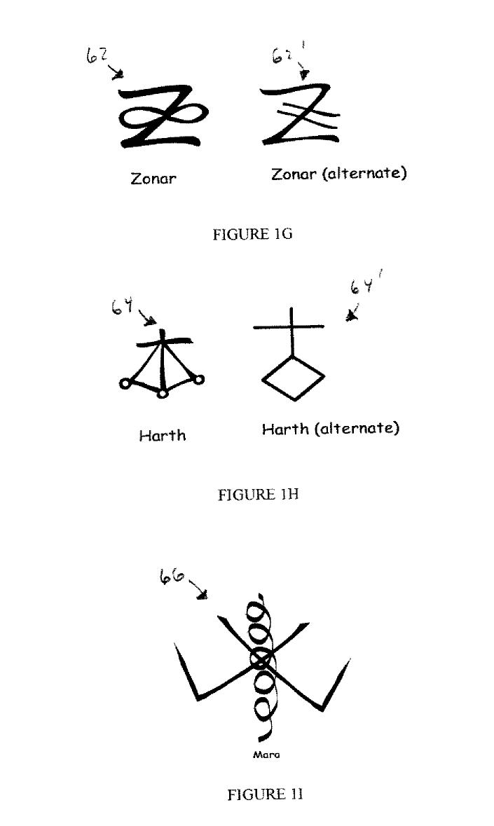

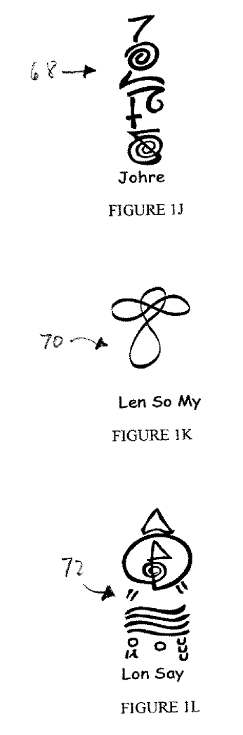

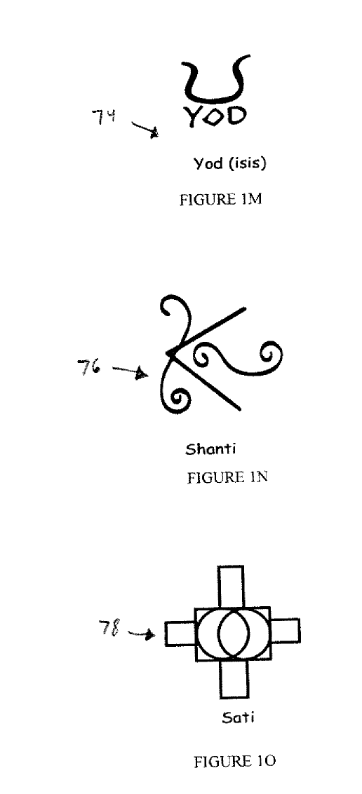

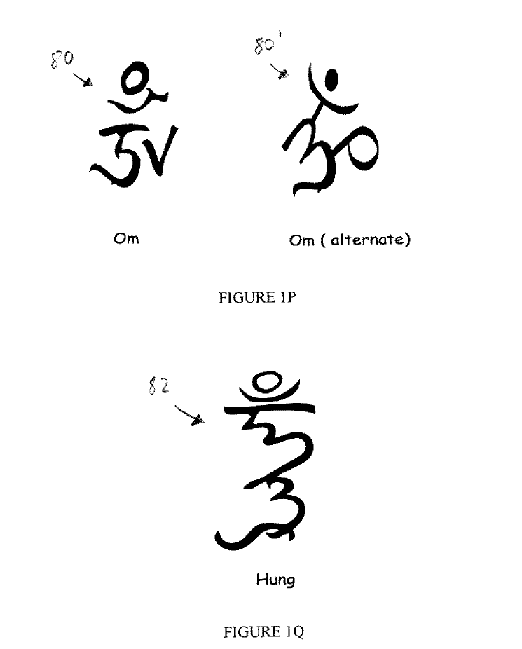

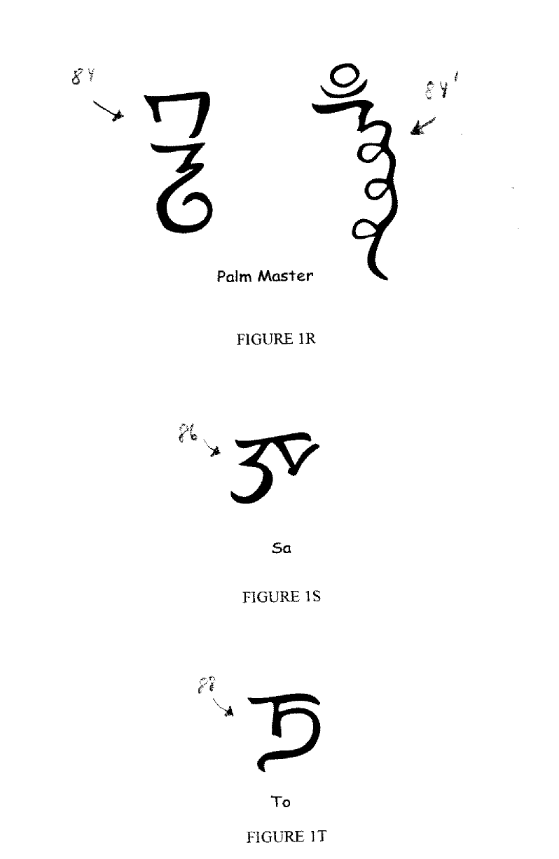

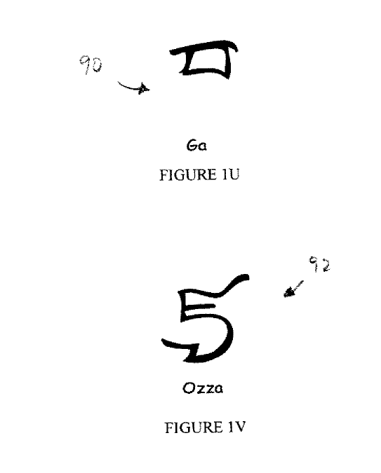

Referring to FIGS. 1A-1V, illustrated therein are various Reiki wellness symbols as they may appear on an article made in accordance with the subject invention. The symbols should be drawn accurately for optimal effect, but there are slight variations to the symbols.

Illustrated in FIG. 1A is a depiction of the Cho Ku Rei symbol 50, together with a backwards variant 50′ of the symbol. The Cho Ku Rei symbol 50 and its variant 50′ may put power behind the well intended thoughts of the user who is then making a positive statement about himself or herself. This symbol 50, 50′ is most effective in one who adds affirmations of well being. It is used to increase power, or current and can mean “put the power here”. The symbol 50, 50′ draws energy from around the user and brings it to focus where the user wants it and to create strength. The symbol 50, 50′ has also been found to remove blocks on a physical level.

Illustrated in FIG. 1B is a depiction of the Sei He Ki symbol 52, together with backwards 52′, upside down 52″, and upside down and backwards 52′″ variants of the symbol. The Sei He Ki symbol 52, 52′, 52″, 52′″ is a gentle and loving symbol helpful in uncovering and clearing the issues found at the root of the user's inner conflict and personal upset. The Sei He Ki symbol 52, 52′, 52″, 52′″ has been found useful for facilitating the removal of emotional blocks which equates to better health and wellness. This symbol 52, 52′, 52″, 52′″ balances the left and right hemispheres of the brain; heals the mind/body connection; releases mental or emotional stresses and brings them to the surface gently; promotes peace and harmony which can enhance memory and increases compassion for self and others.

Illustrated in FIG. 1C is a depiction of the Hon Sha Ze Sho Nen symbol 54, together with two variants 54′, 54″. The Hon Sha Ze Sho Nen symbol 54, 54′, 54″ is a powerful symbol used to transmit to others thoughts of positivity and goodwill to all who receive it. This symbol symbol 54, 54′, 54″ ensures that these thoughts can travel endless miles to the recipient. It 54, 54′, 54″ can also allow a very deep shift to occur with past events that may have had shocking elements to them.

Illustrated in FIG. 1D is a depiction of the Dai Ku Myo symbol 56, together with five variants 56′, 56″, 56′″, 56″″, 56′″″. The Dai Ku Myo symbol 56, 56′, 56″, 56′″, 56″″, 56′″″ is master symbol of honor, integrity, moral and ethical power. The symbol 56, 56′, 56″, 56′″, 56″″, 56′″″ is attuned to the laws and principles of Creation causing the user to have keener insight and sparking their inner wisdom thus facilitating change in their life. The symbol 56, 56′, 56″, 56′″, 56″″, 56′″″ has also been found to facilitate great courage to make changes and shifts to follow the user's heart.

Illustrated in FIG. 1E is a depiction of the Raku symbol 58, together with two lightning bold variants 58′, 58″, and two serpent variants 58′″, 58″″. The Raku symbol 58, 58′, 58″, 58′″, 58″″ holds the secret to the path of personal enlightenment. The user's consciousness is raised and experiences increased vitality, energy and clarity of mind. The symbol 58, 58′, 58″, 58′″, 58″″ has been found to be great for those who need refreshment or tend to feel sluggish, bored or depressed. The symbol 58, 58′, 58″, 58′″, 58″″ is great for grounding energy and raises the kundalini and brings energy throughout the circuits in the user's body.

Illustrated in FIG. 1F is a depiction of the Halu symbol 60. The Halu symbol 60 is used to clear unwanted thoughts and emotional patterns that hold us back from realizing our goals. It is often used as a window to open the user to the present moment of now where the past filled with patterns is no longer holding the user, who can then move forward in a new pattern of health.

Illustrated in FIG. 1G is a depiction of the Zonar symbol 62, together with a variant 62′. The Zonar symbol 62, 62′ is used for unexplained issues. It can facilitate great releases of emotions.

Illustrated in FIG. 1H is a depiction of the Harth symbol 64 together with a variant 64′. This symbol 64, 64′ is used magnify love, truth, beauty, harmony and balance in the heart area. This puts ease and lightness back into the daily dealings of life and helps to release one from harmful feelings of heaviness from depression and self hatred.

The Mara symbol 66 is illustrated in FIG. 1I. This symbol 66 is the flow of the 4 elements in the spiral coning into the male and female elements symbolized by the 2 “v” lines. The symbol 66 triggers the flow of energy coming into the Kundulini awakening the male and female energies. The use of the Mara symbol 66 would be particularly effective with the traditional symbols used to balance the body.

Illustrated in FIG. 1J is a depiction of the Johre symbol 68. The symbol 68 adds light to this area. The vibration of Light is known to raise the vibration of areas that are sluggish and not moving with ease. It gives thought to higher purpose.

The Len So My symbol 70 is depicted in FIG. 1K and adds unconditional love to the aura and leaves the user feeling a lightness, due to the release of self hatred and feelings of unwantedness. It is a gentle symbol that would be effective on pyjamas where the wearer would be cocooned in a gentle wise loving vibration.

Illustrated in FIG. 1L is a depiction of the Lon Say symbol 72. The Lon Say symbol 72 is the symbol of balance. It works with the 4 elements, the air, water, earth, fire. The Lon Say symbol 72 may be depicted with other symbols and may release negativity.

The Yod symbol 74 is illustrated in FIG. 1M and can be used with or without the inclusion of the “YOD” word. This symbol 74 can connect the heart to higher purpose. The depiction of the Yod symbol 74 together with other symbols will result direct improved thinking and correct action.

FIG. 1N depicts the Shanti symbol 76. The Shanti symbol 76 is a symbol used for peacefulness. The symbol 76 evokes a sense of inner peace and allows the release of anxiety. It facilitates relaxation, rest and sleep which in turn facilitate healing.

Illustrated in FIG. 1O is a depiction of the Sati symbol 78. This symbol 78 is used to ground the 4 elements with the male and female energies (represented by the 2 circles). This symbol 78 improves balance and allows for the user who is working on creative projects to bring them to completion. The symbol 78 facilitates the flow of creativity which is needed everyday at work.

The Om symbol 80, together with a variation 80′ are depicted in FIG. 1P. The Om symbol 80, 80′ may be depicted near the user's head to bring in clear thoughts and direct wisdom. Most illness is brought about by incorrect thinking and unwise thought patterns, this symbol 80, 80′ when depicted in combination with other symbols can have a great clearing and relieve foggy thinking.

Also called Om Benza Satto, the Hung symbol 82 depicted in FIG. 1Q is effective in the transmutation of wrong thinking into correct thinking. The symbol 82 may eliminate hard to remove thoughts that are becoming unhealthy physical pains and aches.

Illustrated in FIG. 1R is a depiction of the Palm Master symbol 84, together with a variant 84′. This symbol 84 is also know as the palm Di Ko Mio and is effective in energizing the user's system. This symbol 84 is effective when the body is free from blocks and the user is meditating or studying—in such instance, the user's body can become energized and absorb information better and faster.

The Sa symbol 86, as depicted in FIG. 1S is particularly effective when positioned near the solar plexus area and brings in information. The Sa symbol 86 is for opening the channels of higher study.

Illustrated in FIG. 1T is a depiction of the To symbol 88, which may be used for the enhancement of the Sa symbol 86 and may be positioned proximate the belly area. This symbol may be used for manifesting and releasing fears of wealth, artistic endeavors and self worth. With the Sa symbol 86, the user is able to think or feel his or her purpose, as contrasted with the To symbol 88 which assists the user in manifesting or building his or her purpose.

The symbol Ga 90 is depicted in FIG. 1U. This symbol 90 may be used to capture the positive and direct it into the root or the manifestation of a higher purpose.

FIG. 1V depicts the Ozza symbol 92, which is a symbol of higher thoughts. With the use of this symbol 92, the user brings in correct thinking especially into the lower belly and the root. This affects a person's ability to feel stability and trust; increasing the energy in this area allows creativity to flow.

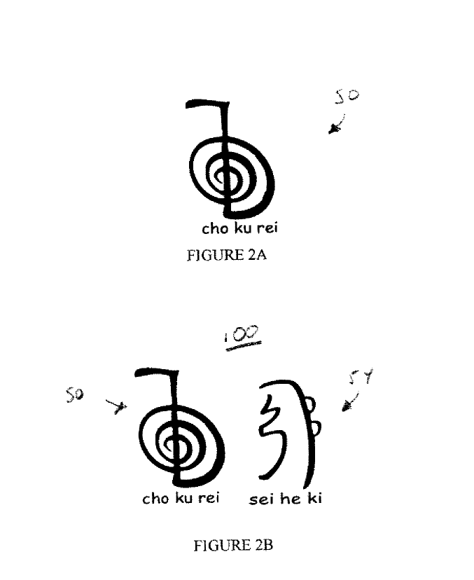

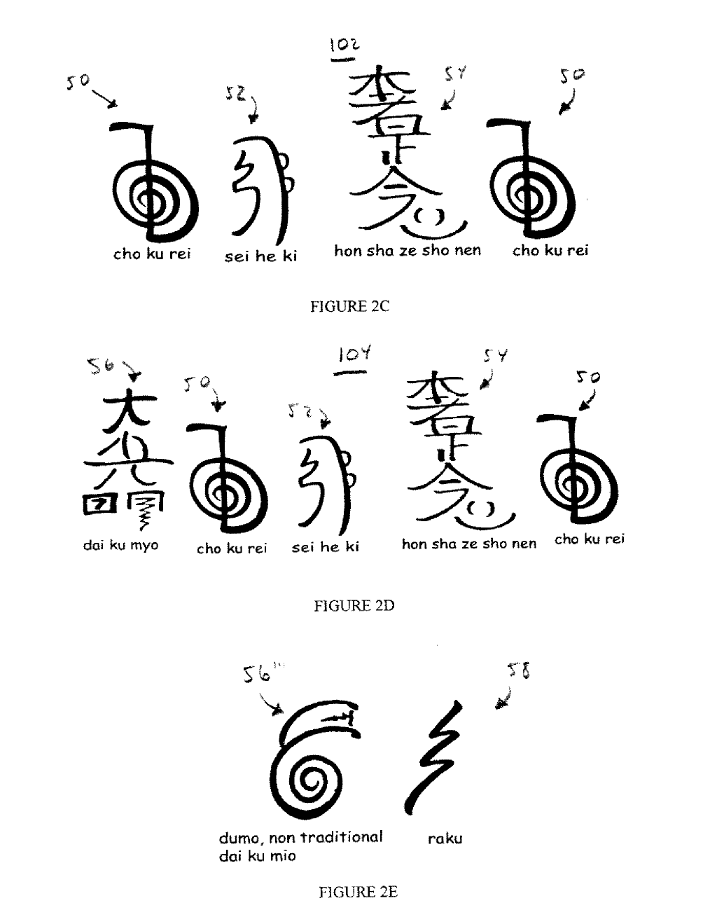

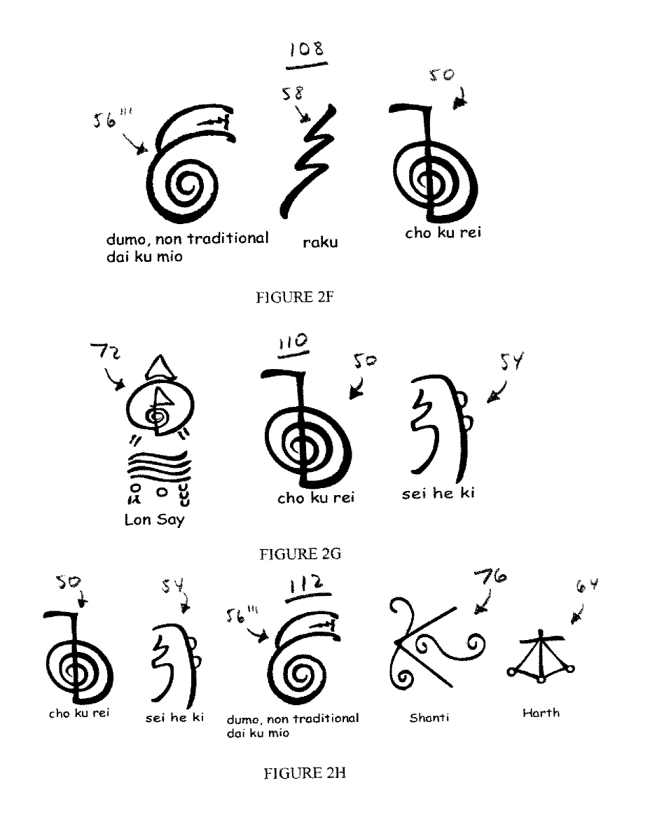

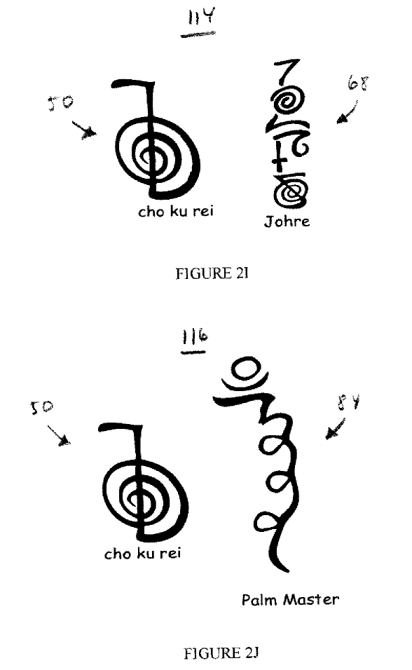

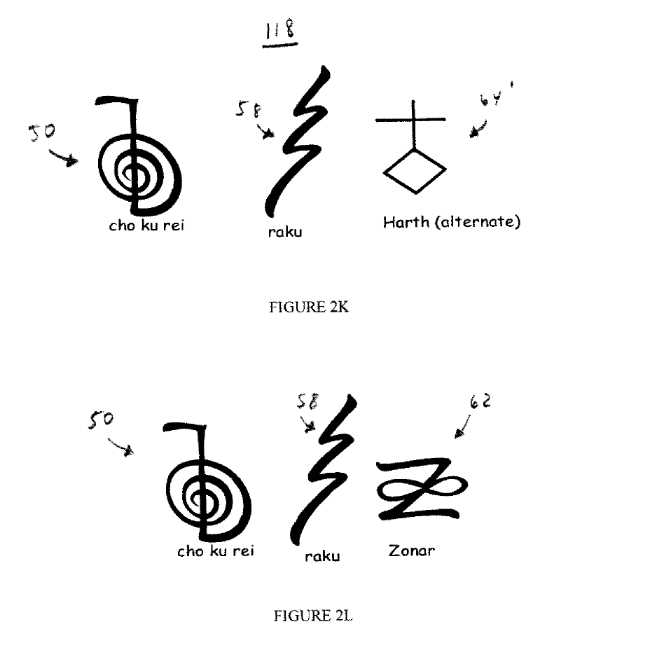

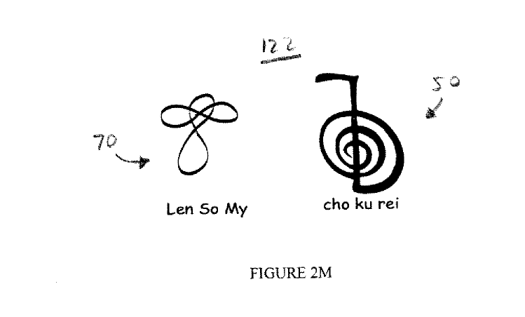

The general effectiveness of the Reiki wellness symbols can be enhanced by placement on the article and the combining with other of the Reiki wellness symbols. The illustrations in FIGS. 2A to 2M illustrate particularly effective wellness indicia including combinations of Reiki wellness symbols.

As noted above, the use of the Cho Ku Rei symbol 50 alone, as illustrated in FIG. 2A enhances balance and overall energies of the user.

The combination 100 of the Cho Ku Rei symbol 50 and the Sei he Ki symbol 54, as illustrated in FIG. 2B, provide a balancing of the emotions, the physical and the unblocking of emotional issues on a physical level which would leave a person feeling physically lighter, more focused, relaxed, balanced and in harmony.

The combination 102 illustrated in FIG. 2C of the Cho Ku Rei symbol 50 and the Sei he Ki symbol 52, the Hon Sha Ze Sho Nen symbol 54 and a second Cho Ku Rei symbol 50 would allow the wearer or user to release emotionally and physically from past issues and hurts or traumas that they have been carrying around most of their lives. This would often mean long term physical issues would start to improve tremendously as negative thinking patterns would be corrected.

FIG. 2D illustrates a combination 104 of 5 symbols, adding the Dai Ku Myo symbol 56 to the combination 102 illustrated in FIG. 2C, which increases the power and releases an insight to the user's healing. Usually, users will be able to make decisions based with honor such as relationship issues, and health issues may no longer be as contentious.

The combination 106 illustrated in FIG. 2E of the non-traditional Dai Ku Mio symbol 56′″ and Raku symbol 58 combined will raise the life force to create abundance of energy to be used for a higher purpose such as creative endeavors. It creates focus and drive. This combination 106 is great for depression and the inability to focus and helps to relieve sadness.

As illustrated in FIG. 2F, the combination 108 adds the Cho Ku Rei symbol 50 to the combination 106 of FIG. 2E and balances the abundant energies and brings them into the physical which would be beneficial for someone with a busy schedule. It also creates a feeling of balance, harmony and good health.

Illustrated in FIG. 2G is a combination 110, in which the Lon Say symbol 72 has been added to the Cho Ku Rei symbol 50 and the Sei he Ki symbol 54. This combination 110 grounds and strengthens the energies of the 4 elements. Humans live on the earth, and to be compatable with the environment would be helpful. The combination 110 may result in improved breathing or the soothing of intestinal issues for the user.

FIG. 2H illustrates the combination 112 of the power symbol, Cho Ku Rei symbol 50, with the emotional Sei he Ki symbol 54, and the grounding master symbol, the non-traditional Dai Ku Mio symbol 56′″, together with the Shanti symbol 76 and the Harth symbol 64 would add great strength to the shift. The release of deep seated fears and trauma provided with the Shanti symbol 76 then great unconditional love and divine peace from the Harth symbol 64 would be grounded with the traditional Reiki symbols. This combination 112 would be particularly effective for someone who has had sleep disorders, hayfever, phobias and other such disorders.

The combination 114 of the Johre symbol 68 together with the Cho Ku Rei symbol 50 is illustrated in FIG. 2I. The Johre symbol 68 would bring in great white light and a soothing feeling from the head down while the Cho Ku Rei symbol 50 would draw it down into the lower body. One particular embodiment would involve the Johre symbol 68 on a user's shirt on the back along the spinal column and the Cho Ku Rei symbol 50 would be on the bottom of the shirt along the hem or on the user's pants. This combination 114 would be effective for someone bed ridden, needing more energy or someone studying needing inspiration and sitting for long periods.

FIG. 2J illustrates another effective combination 116. The Swirling energies of the Palm Master symbol 84 into all the energy centres of the user's body would effectively be made stronger, more stable and lasting with the depiction of the cho ku rei symbol 50. This combination 116 would benefit someone who is already very physically active and wants to enhance their performance or someone who is blocked and suffering from a sluggish system and needs a kick start.

Illustrated in FIG. 2K is yet a further combination 118. The cho ku rei symbol 50 and the raku symbol 58 bring in strong energies to release blocks and clear areas combined with the Harth symbol 64′ it brings in unconditional love with a higher purpose. If used in meditation when a user is looking for answers to personal questions of growth usually answers are forthcoming or the craving for unhealthy addictive substances can be released.

FIG. 2L depicts an additional combination 120. The addition of the Zonar symbol 62 to the cho ku rei symbol 50 and the raku symbol 58 clears difficult-to-remove or hidden issues that lie dormant and unseen. The root causes of issues are often not obvious and used with the Han Sha Ze Sho Nen added to this combination 120 would make it even stronger. This combination would be effective for someone with a illness that stems from repressed sexual issues, family abuse and drug issues.

Illustrated in FIG. 2M is a combination 122 of the Len So My symbol 70 with the Cho Ku Rei symbol 50. Unconditional love with strength and clearing would create a beautiful feeling of acceptance, self worth, respect and generally a balanced sense of self for the user.

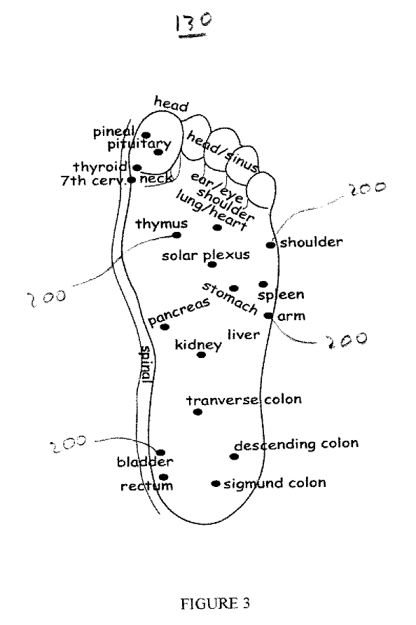

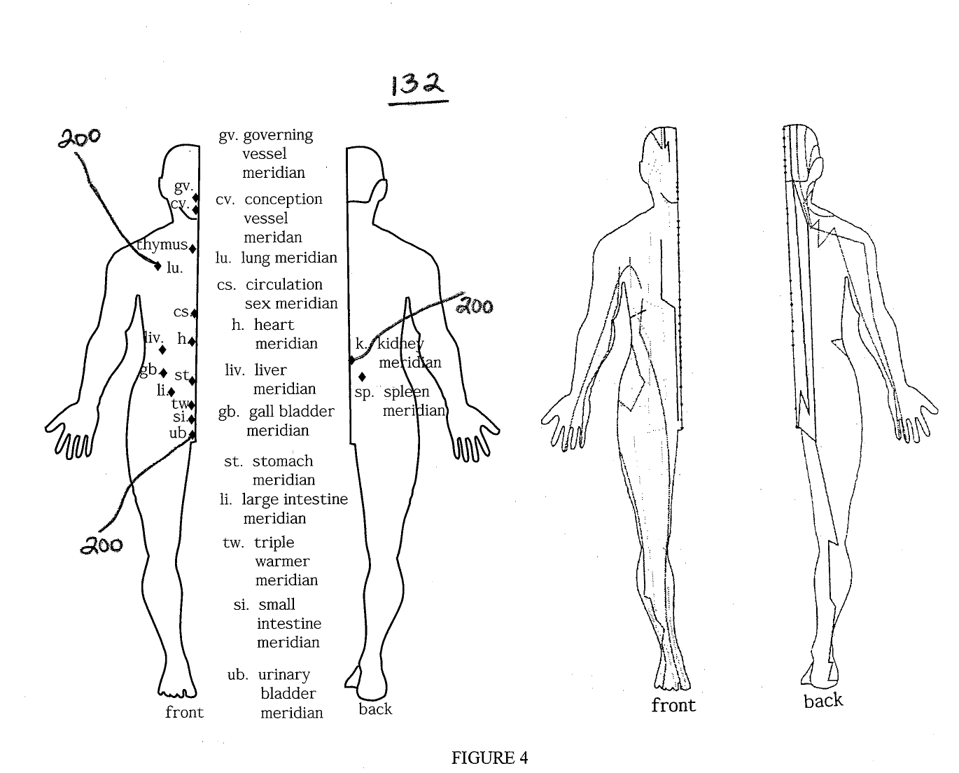

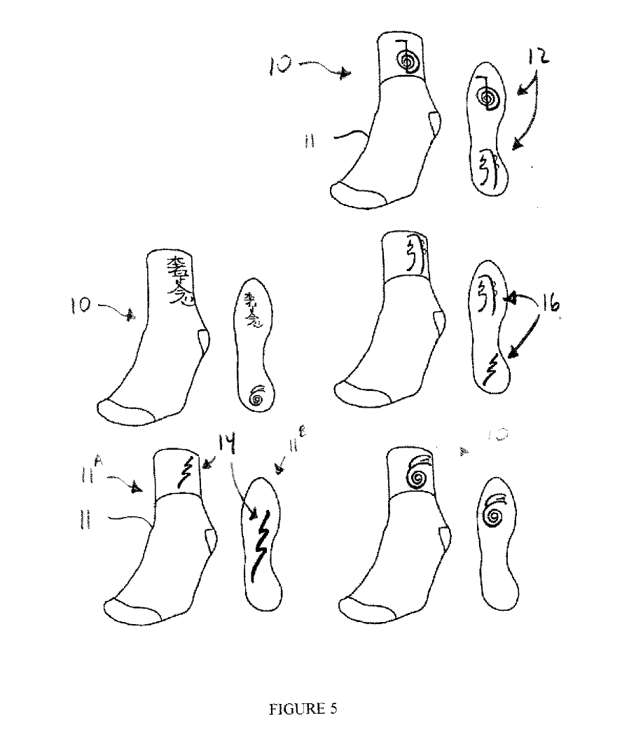

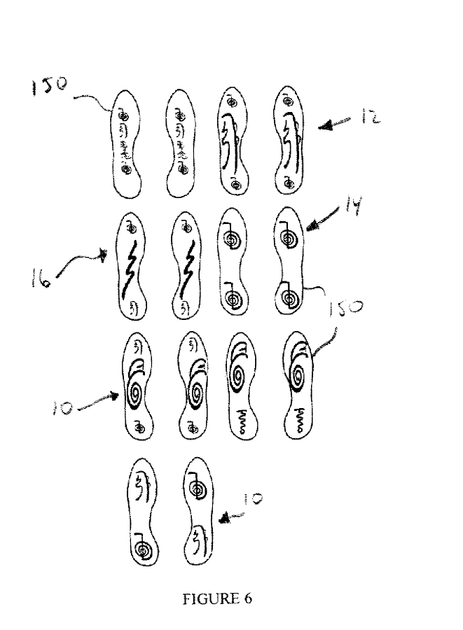

Referring now to FIGS. 3 and 4, illustrated therein are charts of the bottom of the foot 130 (FIG. 3) and of the human body 132 (FIG. 4), indicating various reflexology points 200 (meridians, nadis and chakras) which are understood to affect the energy levels of the body's various organs and systems. For articles 10 intended to be worn by a body (human or animal) or used in close contact with a body, the wellness indicia 12 may preferably be displayed proximate an appropriate reflexology point, or proximate the system or organ desired to be affected. Accordingly, for heart conditions, an article 10 of clothing such as a shirt having a wellness indicia 12 displayed on the chest portion of the shirt, may be worn. Similarly for conditions affecting the head, hats or head scarves having wellness indicia 12 displayed thereon may be worn by the individual.

Referring back to FIGS. 3 and 5 and socks 11 with reiki symbols 14, 16 on them are influencing the wearer through the reflexology points 200 (meridians, nadis and chakras) present in the feet. Similarly, the insoles 150 illustrated in FIG. 6 also have wellness indicia 12 in the form of various single 14 or combinations 16 of the reiki symbols. The combinations and placement of the symbols 14, 16 are important as explained above in relation to FIGS. 1A-1V and 2A-2M with the description of the symbol combinations 16. The symbols 14, 16 may be marked on the insole 150 (and insole of the socks 11) in various ways such as printing, stamping and or embossing, and further in the case of the socks 11 may alternatively be embroidered, embellished or knitted into the fabric. The use of the symbols on the insole is to increase wellness by activating all the meridians in the feet When these reflex points are activated it makes the insoles more effective because they are directed into particular points of receptivity in the body that corresponds to organs and glands. This will enhance the said function of these organs and glands (see charts—FIGS. 3 & 4).

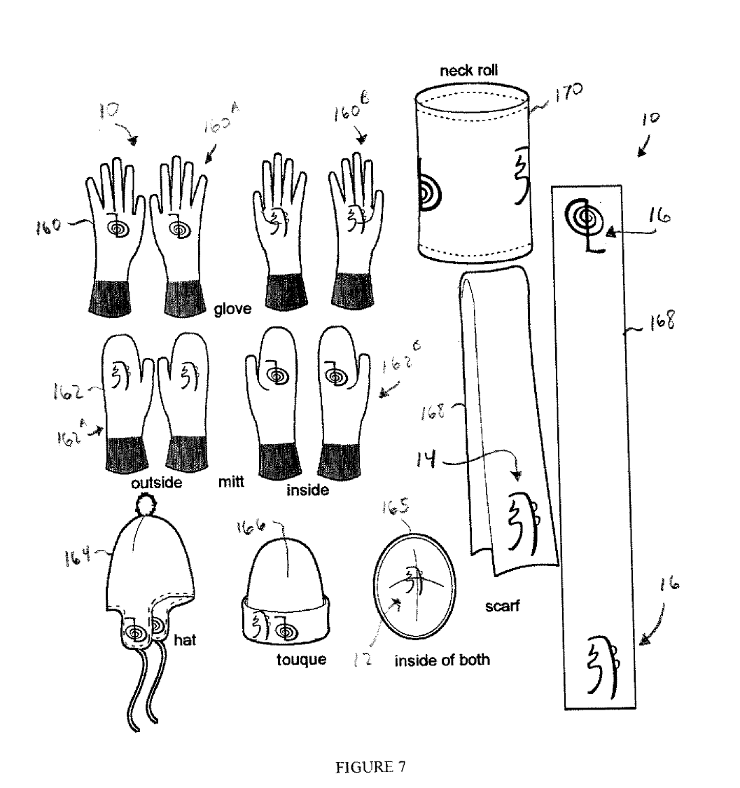

Referring now to FIG. 7, illustrated therein are example articles 10 made in accordance with the subject invention, of clothing including gloves 160 (shown from top view 160^{A} and bottom view 160^{B}), mitts 162 (shown from top view 162^{A} and bottom view 162^{B}), a hat 164, a toque 166 (with an inside view 165 which may correspond to either or both of the toque 166 and hat 164), scarves 168, and a neck roll 170. Each article 160, 162, 164, 166, 168, 170 displays wellness indicia 12, and may display either a single symbol 14 or a combination of symbols 16. As will be understood, for all articles 10 (including those examples illustrated and discussed herein), the wellness indicia 12 is marked and displayed thereon typically by printing, stamping, embossing, knitting, weaving, embossing, embellishing or by using other techniques appropriate in view of the nature of the article 10.

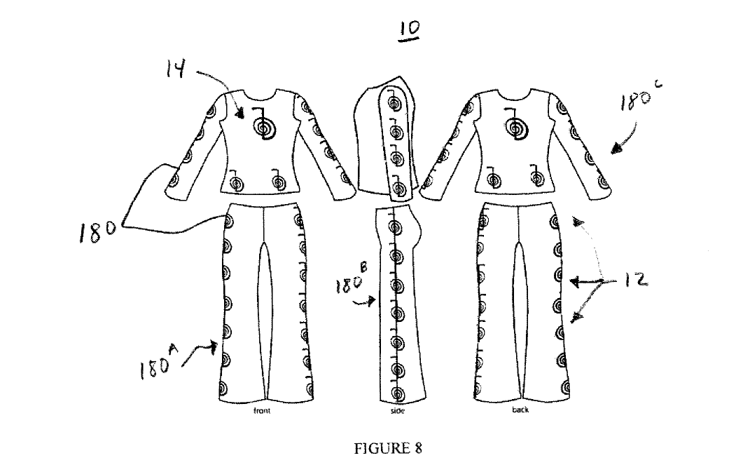

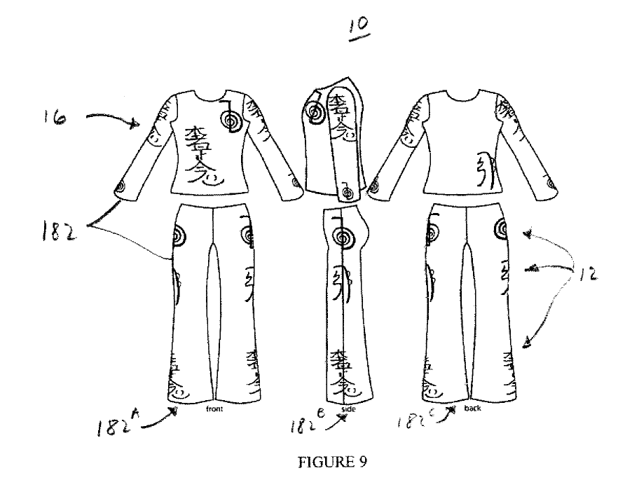

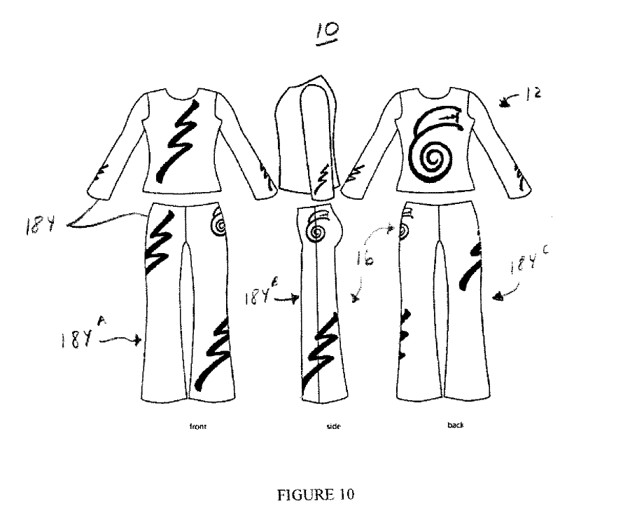

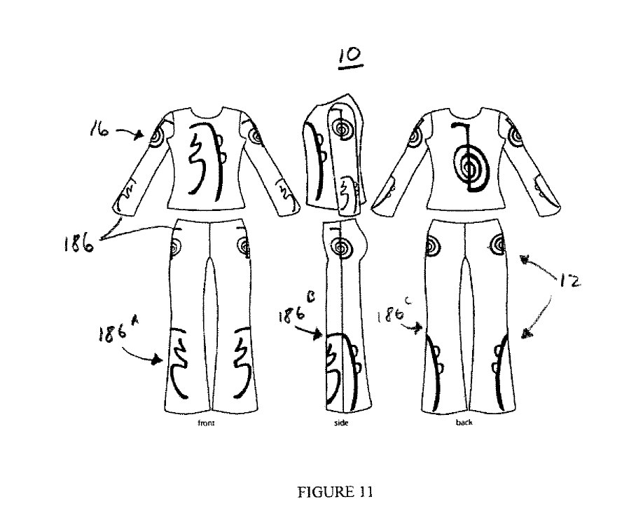

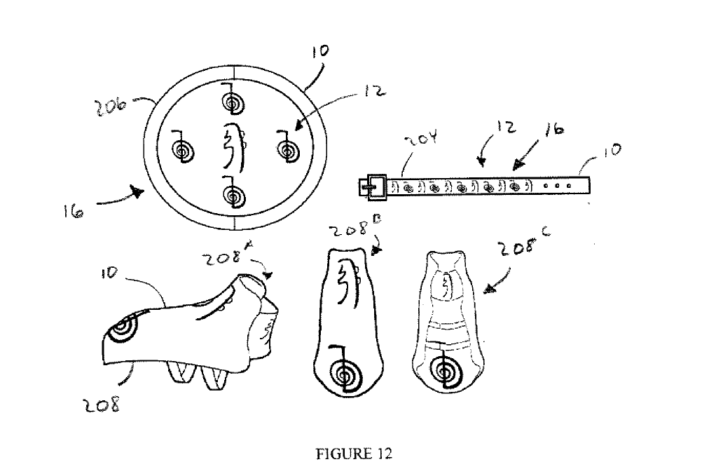

FIGS. 8 to 11 illustrate several articles 10 in the form of outfits 180, 182, 184, 186 (shown from front view 180^{A}, 182^{A}, 184^{A}, 186^{A}, side view 180^{B}, 182^{B}, 184^{B}, 186^{B}, and rear view 180^{C}, 182^{C}, 184^{C}, 186^{C}, having matching shirts 188, 190, 192, 194 and pants 196, 198, 200, 202. Each article 180, 182, 184, 186 displays wellness indicia 12, which may include either a single symbol 14 or a combination of symbols 16. Preferably, the symbols 14, 16 are displayed in an aesthetically pleasing manner.

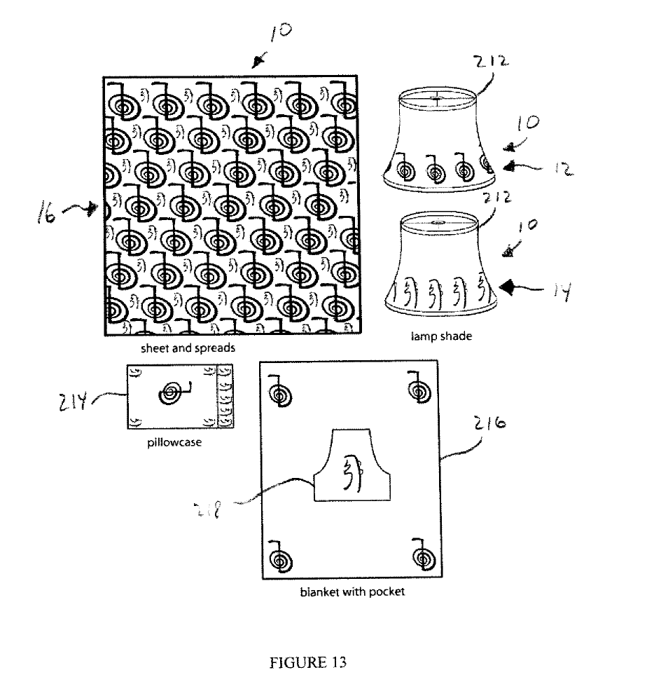

Turning now to FIG. 12, illustrated therein are various other examples of articles 10 made in accordance with the present invention and typically intended for use with animals. As illustrated, wellness indicia 12 may be displayed effectively on collars 204, animal beds or bedding 206, and animal outfits 208 (illustrated from a side perspective view 208^{A}, top view 208^{B}, and bottom view 208^{C}. In these examples 204, 206, 208, the reiki symbols are displayed in combinations 16.

FIG. 13 displays yet other examples of articles 10 made in accordance with the present invention, including a sheet or bedspread 210, lamp shades 212, a pillowcase 214, and a blanket 216 having a pocket 218. Each such article 210, 212, 214, 216 displays wellness indicia 12, in the form of a reiki symbol 14 which may be repeated, or a combination of symbols 16.

It should be understood that while various combinations of wellness symbols 16 have been illustrated in FIGS. 2A-2M, other combinations of wellness symbols or variations of the wellness symbols may be used. Similarly, the locations of the symbols on articles, particularly on articles such as clothing or bedding which are intended for contact with an individual's body, may be adjusted at least in part to correspond more effectively the body's reflexology points of particular interest. Furthermore, while a wide range of articles 10 has been illustrated and described herein, it should be understood that the illustration and listing of such articles 10 is not intended to be exhaustive and other types of articles, or similar articles having different appearances may be created in accordance with the present invention. As should be understood, it is preferable if the combination of symbols 16 is selected and the location of each (corresponding to appropriate reflexology points) is determined based on the desired effect for the user (or wearer).

Thus, while what is shown and described herein constitute preferred embodiments of the subject invention, it should be understood that various changes can be made without departing from the subject invention, the scope of which is defined in the appended claims.

I claim:

1. An article intended to be positioned proximate a body, the article comprising wellness indicia displayed thereon.

2. The article as claimed in claim 1 comprising an article of clothing.

3. The article of clothing as claimed in claim 2, wherein the article of clothing is configured to be worn on the body of an individual.

4. The article of clothing as claimed in claim 3, wherein the individual has at least one meridian point and wherein the wellness indicia is positioned proximate a reflexology point of the individual's body.

5. The article of clothing as claimed in claim 3, wherein the wellness indicia is positioned proximate the individual's heart.

6. The article of clothing as claimed in claim 3, wherein the wellness indicia comprises at least one Reiki symbol.

7. The article of clothing as claimed in claim 6, wherein the at least one Reiki symbol is selected from the set of symbols consisting of: Cho Ku Rei, Cho Ku Rei backwards, Sei He Ki, Sei He Ki, Sei He Ki backwards, Sei He Ki upside down, Sei He Ki upside down and backwards, Hon Sha Ze Sho Nen, Dai Ko Myo, Raku, Halu, Zonar, Harth, Mara, Johre, Len So My, Lon Say, Yod, Shanti, Sati, Om, Hung, palm Di Ko Mio, Sa, To, Ga, Ozza.

8. The article of clothing as claimed in claim 5, wherein the set of symbols further consists of variations of each symbol in the set.

9. The article of clothing as claimed in claim 1, wherein the wellness indicia comprises a plurality of Reiki symbols.

10. The article of clothing as claimed in claim 9, wherein the plurality of Reiki symbols are selected from the set of symbols consisting of: Cho Ku Rei, Cho Ku Rei backwards, Sei He Ki, Sei He Ki, Sei He Ki backwards, Sei He Ki upside down, Sei He Ki upside down and backwards, Hon Sha Ze Sho Nen, Dai Ko Myo, Raku, Halu, Zonar, Harth, Mara, Johre, Len So My, Lon Say, Yod, Shanti, Sati, Om, Hung, palm Di Ko Mio, Sa, To, Ga, Ozza.

11. The article of clothing as claimed in claim 10, wherein the set of symbols further consists of variations of each symbol in the set.

12. The article as claimed in claim 1, wherein the wellness indicia comprises at least one Reiki symbol.

13. The article as claimed in claim 12, wherein the at least one Reiki symbol is selected from the set of symbols consisting of: Cho Ku Rei, Cho Ku Rei backwards, Sei He Ki, Sei He Ki, Sei He Ki backwards, Sei He Ki upside down, Sei He Ki upside down and backwards, Hon Sha Ze Sho Nen, Dai Ko Myo, Raku, Halu, Zonar, Harth, Mara, Johre, Len So My, Lon Say, Yod, Shanti, Sati, Om, Hung, palm Di Ko Mio, Sa, To, Ga, Ozza.

14. The article as claimed in claim 13, wherein the set of symbols further consists of variations of each symbol in the set.

15. The article as claimed in claim 14, wherein the article is selected from the set consisting of: socks, insoles, shirts, pants, gloves, hats, touques, scarves, neck rolls, lingerie, support wraps, dishes, water containers blankets, collars, animal clothing, bedding, wallpaper, lampshades, sheets, pillow cases.

16. The article as claimed in claim 12, wherein the wellness indicia comprises a plurality of Reiki symbols.

17. The article as claimed in claim 15, wherein the plurality of Reiki symbols are selected from the set of symbols consisting of: Cho Ku Rei, Cho Ku Rei backwards, Sei He Ki, Sei He Ki, Sei He Ki backwards, Sei He Ki upside down, Sei He Ki upside down and backwards, Hon Sha Ze Sho Nen, Dai Ko Myo, Raku, Halu, Zonar, Harth, Mara, Johre, Len So My, Lon Say, Yod, Shanti, Sati, Om, Hung, palm Di Ko Mio, Sa, To, Ga, Ozza.
